(19) United States
(12) Patent Application Publication
(10) Pub. No.: US 2006/0135848 A1
(43) Pub. Date: Jun. 22, 2006

(54) Endoscope guiding device

(75) Inventors: Drake Lansdon Koch, Kansas City, MO (US); Michael J. Armentrout, Leawood, KS (US); William A. Hartong, Leawood, KS (US); Gene Lee Armentrout, Kansas City, MO (US)

Correspondence Address: Kenneth W. Iles, 9903 West 129th Street, Overland Park, KS, 66213-3222, US

(73) Assignee: Zutron Medical, L.L.C., Kansas City, MO (US)

(21) Appl. No.: 11/159,566

(22) Filed: Jun. 22, 2005

Related U.S. Application Data
(63) Continuation-in-part of application No. 11/021,524, filed on Dec. 22, 2004.

(51) Int. Cl. A61B 1/00

(52) U.S. Cl. 600/104; 600/144

(57) ABSTRACT

An endoscope guiding device has a main wire body made of nitinol memory wire to retain its shape after bending, a handle fixed to the proximal end of the wire body and, at the distal end a rounded tip and adjacent to the distal end a reduced diameter end portion and a transition zone leading from the reduced diameter end portion to the larger diameter of the main body portion of the wire body. The rounded tip, reduced diameter portion and the transition zone are formed by centerless grinding. The main wire body may have additional reduced stiffness, that is, reduced diameter, portions at any desired point along its length in addition to the reduced diameter portion adjacent to the distal end. The guiding device is inserted into an endoscope and stiffens the endoscope sufficiently to allow the endoscope to be guided, for example, through a colon or intestine readily.

### CROSS-REFERENCE TO RELATED APPLICATIONS

This application is a continuation-in-part of U.S. application Ser. No. 11/021,524 having a filing date of Dec. 22, 2004.

### STATEMENT REGARDING FEDERALLY SPONSORED RESEARCH OR DEVELOPMENT

Not applicable.

### SEQUENCE LISTING

Not applicable

### BACKGROUND OF THE INVENTION

The present invention is related to a device for stiffening an endoscope by inserting the device into the endoscope to a point in proximity to the distal end of the endoscope, making guiding the endoscope, into a desired position easier and more accurate. The stiffening device is withdrawn from the endoscope after the endoscope is in its desired location to allow insertion of medical instruments into the endoscope.

### DESCRIPTION OF THE RELATED ART INCLUDING INFORMATION DISCLOSED UNDER 37 C.F.R. 1.97 and 1.98

Endoscopes are used in many different kinds of a medical procedures, including surgery, as an alternative to more invasive procedures. In some cases, the path the endoscope is intended to follow is short and straight. In other cases, however, such as the examination of the colon, small intestine, and particularly the cecum, the pathway is long and circuitous. In particular, in the case of the colon, the pathway typically includes very sharp turns. Most endoscopes are highly flexible and can or tend to bunch up in an accordion-like fashion when an obstacle, such as a turn in the colon, is encountered. If the endoscope is made rigid, the opposite effect occurs—the endoscope will remain straight, but the colon bunches up, retarding the endoscope from reaching the desired location. Although a skilled physician can ultimately locate the distal end 20 of the endoscope 14 in the desired location, this process requires significant skill and time, reducing productivity and increasing patient discomfort.

When the endoscope has reached the desired location within the intestine, it now must be flexible so that the bending tip, which is adjacent to the distal end of the endoscope, can be manipulated into different positions for conducting medical procedures, for example, taking photographs, excising polyps, and so forth. Therefore, a permanently rigid endoscope cannot be employed successfully.

With increasing emphasis on early detection on medical problems in the colon, the use of exploratory endoscopy has increased, but endoscopes for conducting such procedures effectively have not been improved, leading to frustration by the practitioner and difficult assessments of the medical condition of the colon.

At one time, physicians viewed fluoroscope pictures of the colon as an endoscope was advanced. Although this step has proved unnecessary, great skill and patience and substantial time is required for even the most experienced physicians to position the distal end of an endoscope appropriately. If the efficiency of this portion of the procedure could be improved, many more patients could receive the benefit of such examinations.

Some efforts to achieve such an endoscope have led to published patent applications or issued patents.

For example, United States Patent Application No. US 2003/0032859 discloses a guide for insertion into an endoscope, but this guide is limp and is made rigid by control wires. Further it is only about half as long as the endoscope. This is unnecessarily complex and therefore expensive, and cannot stiffen an endoscope over most of its length. Further, the sharp tip at its distal end can damage the main channel of an endoscope.

Similar observations apply to United States Patent Application Publication No. 2002/0120178, which discloses a similar structure and shares an inventor.

Another approach involves an endoscope having a distal end portion with segments can be steered to a limited degree by causing the distal end to move back and forth is disclosed in U.S. Pat. No. 6,468,203, which increases the cost of the endoscope and can only be used as part of an endoscope, that is, the steering mechanism cannot be removed and used on another endoscope. Utilizing this device would require replacing a facility's existing inventory of endoscopes, which is a very substantial investment.

U.S. Pat. No. 6,379,334 discloses wrapping screw threads around the exterior of the distal end of a catheter or the like and basically screwing the catheter up a channel. This approach will not work in examining, for example, a colon or intestine because any channel is of larger diameter than the endoscope, so screw threads would not gain purchase on the side walls of the intestine. A similar system, subject to the same drawbacks, is disclosed in U.S. Pat. No. 5,989,230.

U.S. Pat. No. 5,921.915 discloses an endoscope having a sheath with a distal end that is resilient and bent to direct an instrument in a specific direction. This device also requires purchasing a new endoscope and does not provide the physician with the means for guiding an endoscope over an extended distance.

A separate device that can be inserted into and removed from an existing inventory of endoscopes would overcome many of these problems, be more economical and of greater utility than solutions built into an individual endoscope. Prior art stiffening devices have attempted to utilize piano wire to stiffen an endoscope. Through use, however, it has been determined that normal spring steel wire, such as piano wire, although used previously, is not a desirable material because it retains bends. For example, if the piano wire is formed into a coil, it will tend to retain the shape of the original coil unless it is held under considerable tension. When the tension is released, the piano wire springs back to a coiled condition similar to the original shape.

Further, a wire body of any type of the desired stiffness to guide an endoscope through the intestine is too stiff to pass through the sharp elbow turn of the instrument port of the endoscope with the same feel to the user as the medical instruments that are inserted into and withdrawn from the endoscope, disturbing the user physicians. Further the sharp distal end on a typical wire, formed by making a cut perpendicular to the longitudinal axis of the wire, may easily damage the interior side wall of the endoscope.

Therefore, there is a need for an endoscope guiding device that allows the physician user to guide the endoscope readily along a particular circuitous, flexible and compressible path such as that found in the colon; that allows the endoscope to be guided basically throughout its full length, save for the flexible bending tip portion adjacent to the distal end of the endoscope; that can be used with more than one endoscope; that does not require the acquisition of any additional endoscopes; that can be inserted into and withdrawn from an endoscope by forces similar to the forces required for insertion and removal of diagnostic and surgical instruments commonly used with endoscopes.

### BRIEF SUMMARY OF THE INVENTION

Accordingly, it is a primary object of the present invention to provide an endoscope guiding device that allow the physician user to guide the endoscope readily along a particular circuitous, flexible and compressible path such as that found in the colon.

It is another object of the present invention to provide an endoscope guiding device that allows the endoscope to be guided basically throughout its full length, save for the flexible bending tip portion adjacent to the distal end of the endoscope.

It is another object of the present invention to provide an endoscope guiding device that can be used with more than one endoscope.

It is another object of the present invention to provide an endoscope guiding device that does not require the acquisition of any additional endoscopes.

It is another object of the present invention to provide an endoscope guiding device that can be inserted into and withdrawn from an endoscope by forces similar to the forces required for insertion and removal of diagnostic and surgical instruments commonly used with endoscopes.

These objects are achieved by providing an endoscope guiding device having an elongated nitinol wire body having a diameter of about 1.6 mm with a handle attached to the proximal end, and a round tip on the distal end, with a reduced diameter portion adjacent to the rounded tip distal end to ease insertion into an endoscope and a tapered transition zone portion between the reduced diameter portion and the full sized diameter portion. The reduced diameter portion is preferably about 1.2 mm in diameter and the tapered transition zone is formed by grinding along the length of the transition zone at an angle of about 0.5°-5°, with the preferred angle being 2°. The reduced diameter distal end portion provides the flexibility desirable for inserting the endoscope guiding device into an endoscope, particularly if the port being used incorporates an elbow, as is typically the case, while the full diameter wire body portion imparts a suitable degree of sniffiness to the endoscope (which a 1.2 mm guide does not). The use of nitinol wire for the wire body provides the physician user with a repeatable experience because the resulting wire body does not retain deformations as it passes through various curves and bends, but returns to its exact straight shape and predictable flexibility after removal from the endoscope. Further, the resulting device is easily sterilized for reuse in other procedures.

Other objects and advantages of the present invention will become apparent from the following description taken in connection with the accompanying drawings, wherein is set forth by way of illustration and example, the preferred embodiment of the present invention and the best mode currently known to the inventor for carrying out his invention.

### BRIEF DESCRIPTION OF THE SEVERAL VIEWS OF THE DRAWING

FIG. 1 is a schematic side view of an endoscope guiding device according to the present invention.

FIG. 2 is an enlarged view of the endoscope of FIG. 1 illustrating the insertion and removal of the endoscope guiding device of FIG. 1.

FIG. 3 is an isometric view of an enlarged upper portion of an endoscope illustrating the insertion of the endoscope guiding device of FIG. 1.

FIG. 4 is a side view of the endoscope guiding device of FIG. 1 shown in use in an endoscope with a portion of the endoscope body broken away to show the point of furthest advance of the endoscope guiding device of FIG. 1.

FIG. 5 is a side view of the endoscope guiding device of FIG. 1 shown fully inserted into an endoscope with a portion of the endoscope body broken away to show the point of furthest advance of the endoscope guiding device of FIG. 1, which does not penetrate into the flexible tip bending portion of the endoscope.

FIG. 6 is a front cross-sectional view of a person's intestine illustrating the insertion of an endoscope without the aid of the endoscope guiding device of FIG. 1.

FIG. 7 is a front cross-sectional view of a person's intestine illustrating the insertion of an endoscope with the aid of the endoscope guiding device of FIG. 1.

FIG. 8 is a schematic side view of a test devised to measure the stiffness of an endoscope guiding device of FIG. 1.

FIG. 9 is a systematic side view of the test results of a variety of experiments used to develop a suitable material for the endoscope guiding device of FIG. 1.

FIG. 10 is a side view of the distal end portion of a wire body for use in the endoscope guiding device on FIG. 1, illustrating a desired point of tapering of the wire body of the endoscope guiding device of FIG. 1.

FIG. 11 is a side view of the distal end portion of a wire body for use with the endoscope guiding device of FIG. 1, illustrating a different desired point of tapering of the wire body of the endoscope guiding device of FIG. 1.

FIG. 12 is an enlarged side view of a rounded tip portion of the distal end of the endoscope guiding device of FIG. 1,

FIG. 13 is an isometric view of an alternative embodiment of a handle fixed to the proximal end of the endoscope guiding device of FIG. 1.

FIG. 14 is a side view of the distal end portion of a wire body for use with the endoscope guiding device of FIG. 1 showing an alternative embodiment.

FIG. 15 is an enlarged view of the rounded end distal tip portion of the wire body of FIG. 14.

FIG. 16 is a side view of an alternative embodiment of the wire body for use with the endoscope guiding device of FIG. 1, having a length of weakness created by forming a reduced diameter section between a proximal end and a distal end of the wire body.

FIG. 17 is a side view of an alternative embodiment of the wire body for use with the endoscope guiding device of FIG. 1, having two separate spaced apart lengths of weakness created by forming a reduced diameter section between a proximal end and a distal end of the wire body.

FIG. 18 is a side view of an alternative embodiment of the wire body for use with the endoscope guiding device of FIG. 1 having a reduced diameter distal end formed by a uniformly tapered portion adjacent to a distal end, with the thinnest portion of the reduced diameter portion lying immediately adjacent to the rounded distal end tip.

### DETAILED DESCRIPTION OF THE INVENTION

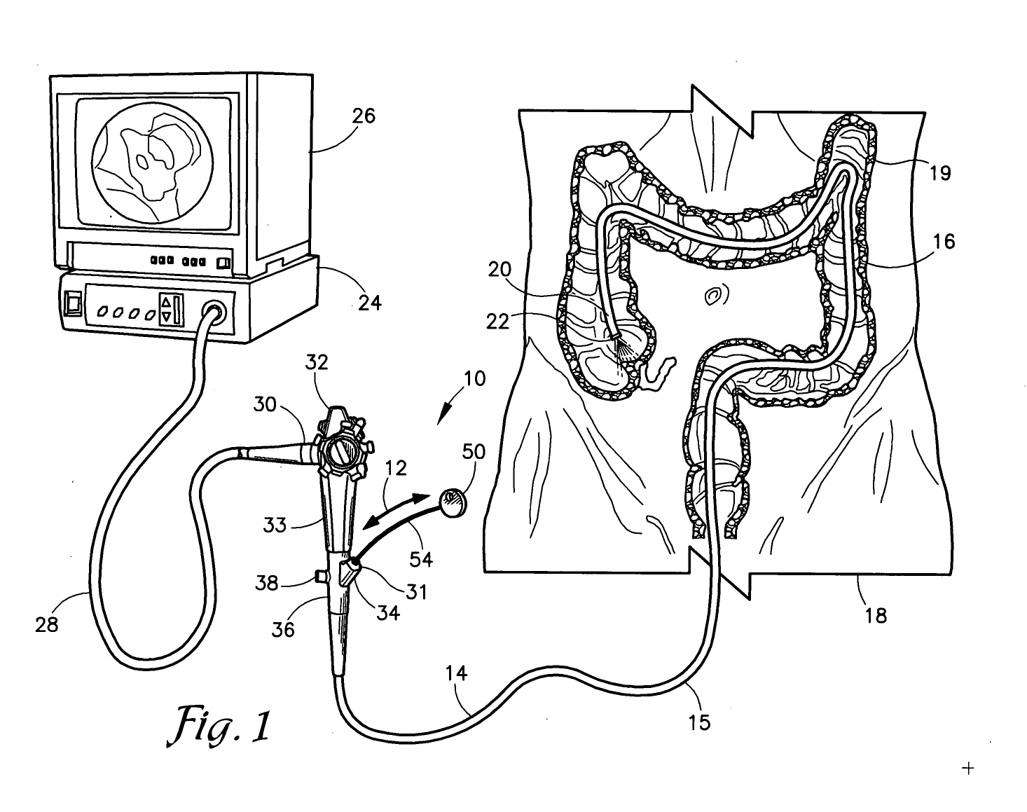

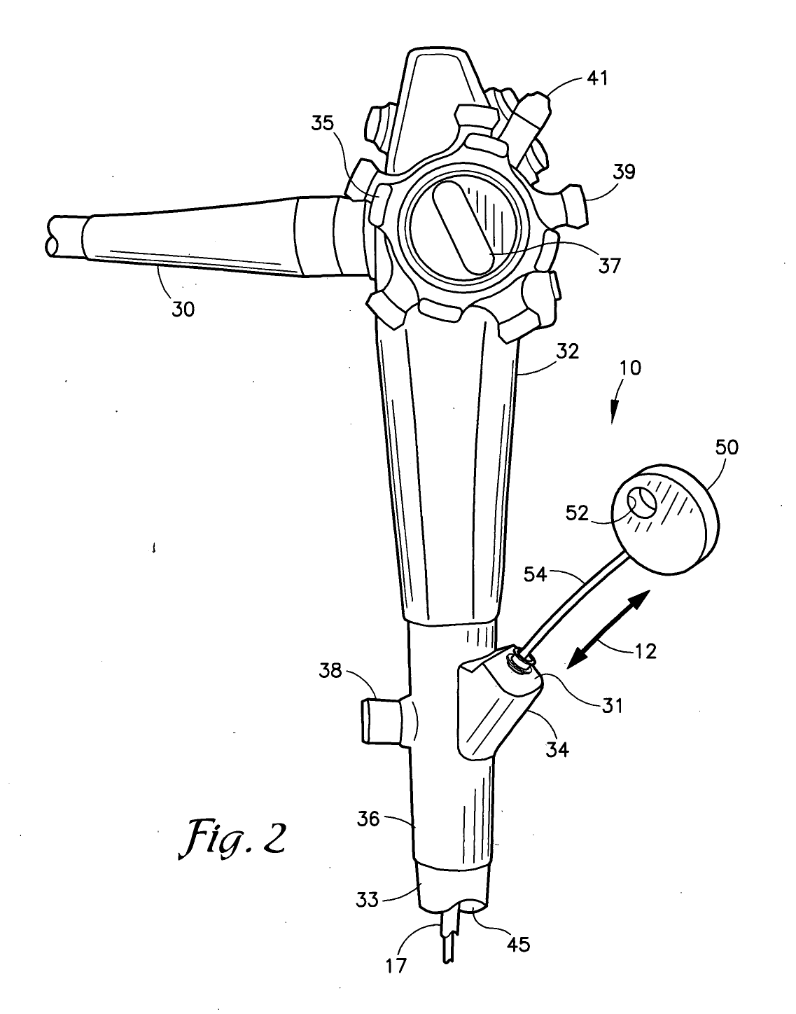

Referring to FIGS. 1, 2, there is shown an endoscope guiding device 10 according to the present invention being inserted along the direction of the leftward pointing head on the double headed arrow 12 into an endoscope 14 that is being guided through the intestine 16 of a patient 18. As shown in FIG. 1, the distal end 20 of the endoscope 14 includes instruments, such as the light and camera assembly 22, which sends signals to a computer 24 and associated monitor 26 through the cable 17 (FIG. 2), which may contain electrical wires, fiber optic cable or other information transmission means in the flexible tubular body 15 of the endoscope 14 and through a cable 28 connected to the computer 24 and to the endoscope 14 by the instrument connector 30, which forms a portion of the head of 32 of the endoscope 14, which is at the proximal end 33 of the endoscope 14. An instrument port of 34 having an upper end 31 lies adjacent to the lower end 36 of the endoscope head 32 and projects outwardly from the general axis of the endoscope head 32 at an angle of approximately 45°. A second instrument port 38 accommodates additional instruments through a separate channel, a feature found in some endoscopes. Once the distal end 20 of the endoscope 14 has reached its intended location, the endoscope guiding device 10 is withdrawn from the endoscope 14 along the a direction indicated by the right-hand head on the double-headed arrow 12.

Referring to FIG. 2, the head 32 of the endoscope 14 includes a four lobe control knob 35 having an associated lock 37, which controls left and right movement of the distal end 20 tip of the endoscope body 15 through tightening or loosening cables inside the endoscope body 15. Located behind the four lobe control knob 35 is a five lobe control knob 39 and associated secondary lock 41, which operate similarly to control up and down motion of the distal end 20 tip, so that the physician can position the working tip, i.e., distal end 20 tip of the endoscope 14 in exactly the position needed for a particular procedure. During use, physicians typically may insert, use, and withdraw many different instruments from the endoscope 14. The endoscope guiding device 10 may be conveniently viewed as an instrument that is inserted into the endoscope 14 for allowing the distal end 20 to be maneuvered easily to the desired location for further examination, and may be used repeatedly during a given procedure, which may involve many stops along the route.

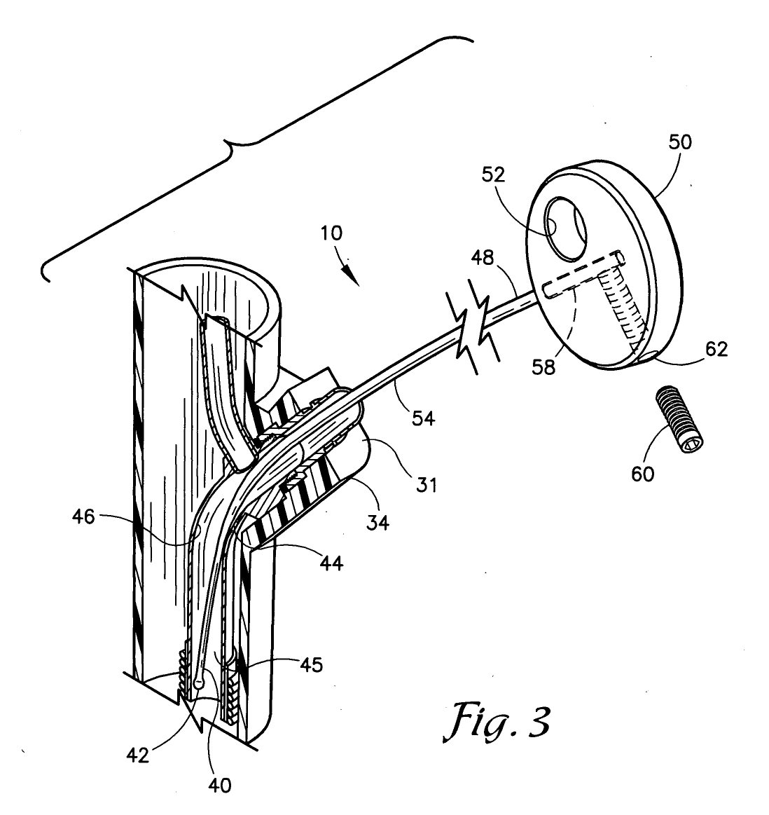

Referring to FIG. 3, the distal end 40 of the endoscope guiding device 10 includes a rounded tip 42, which allows the distal end 40 to be guided through the sharp elbow turn 44, where the instrument port 34 incorporates an elbow to guide any instrument inserted into the instrument port 34 into the main channel of the endoscope 14 through the upper end 31 of the instrument port 34. A blunt or sharp distal end of an endoscope guiding device 10 that, for example, was formed by simply cutting straight across a wire, will scar, puncture or otherwise damage the interior side wall 46 that separates the endoscope proper from the instrument port 34 when it is inserted and may also damage the interior side wall of the endoscope 14, particularly as the endoscope 14 is passed through bends in the intestine 16. Physicians are very sensitive to the feel of the instruments being inserted into and withdrawn from the endoscope 14 and are not comfortable using an endoscope guiding device 10 that has a different feel from other instruments when it is inserted into and withdrawn from the endoscope. A physician knows from experience that any excessive insertion force indicates excessive wear on the inner side wall surfaces 46, which are typically coated with polytetrafluoroethylene. The physical structure of the endoscope guiding device 10 is designed to provide the tactile feedback of medical instruments when it is being inserted into and withdrawn from the endoscope 14.

Still referring to FIG. 3, the proximal end 48 of the endoscope guiding device 10, includes a disk-shaped handle 50, having an off-center bore 52 there through for hanging the endoscope guiding device 10 from a hook or the like for storage. The wire body 54 of the endoscope guiding device 10 is secured to the handle 50 by inserting the proximal end 48 of the wire body 54 into the bore 58 and tightening the set screw 60, which is threadably received in the bore 62, against the proximal end 48 of the wire body 54.

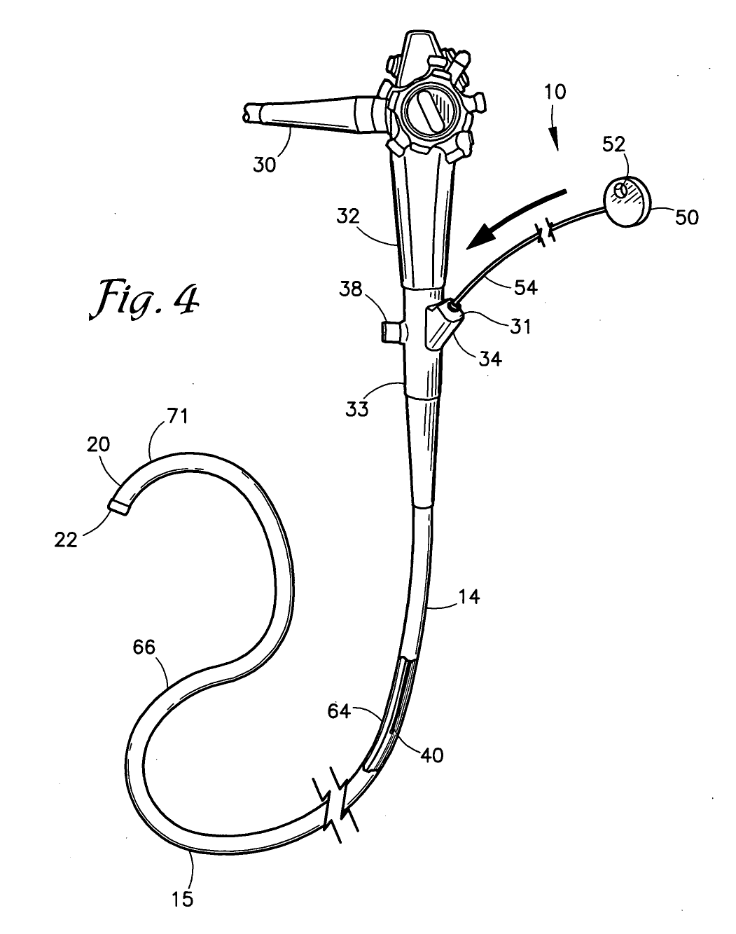

Referring to FIG. 4, the entire endoscope 14 with the endoscope guiding device 10 inserted into it is shown without the environmental clutter associate with illustrating the device inside a patient. As shown, the distal end 40 of the endoscope guiding device 10 has been advanced into the endoscope 14 only part way, namely to the point of furthest advance 64. From the proximal end 33 of the endoscope 14 to the point 64, the flexible tubular endoscope body 15 is relatively stiff, having a gentle curved shape, but from the point of furthest advance 64 to the distal end 20, it is floppy and naturally forms a serpentine shape, shown as the S-curve 66 when the distal end 20 encounters any significant resistance.

Referring to FIG. 5, the distal end 40 of the endoscope guiding device 10 has been advanced to the point 68, that is to the beginning of the flexible bending distal tip portion 71 of the flexible tubular endoscope body 15 adjacent to the distal end 20, providing a stiffening effect that leaves the endoscope body 15 more nearly rigid than before insertion and advancement of the endoscope guiding device 10, but still flexible enough to form the curved portion 70, a capacity required for maneuvering the flexible tubular endoscope body 15 around obstacles and bends in the intestine 16. The degree stiffness imparted to the flexible tubular endoscope body 15 is critical to development of a successful endoscope guiding device 10—too rigid and the endoscope 14 cannot be guided around obstacles and bends in the intestine 16—too limp and the endoscope merely collapses on itself when any obstacle or bend is encountered.

Still referring to FIG. 5, the distal end 40 of the endoscope guiding device 10 must be kept out of the flexible bending distal tip portion 71 because the physician steers the endoscope 14 by manipulating and bending the flexible bending distal tip portion 71 while pushing on the endoscope. In building the endoscope guiding device 10, a wire body 54 element is sized to the length of the particular endoscope 14 being used, and can be made in any desirable length, but in general is about 170 cm (5 feet, 7 inches), which is several centimeters shorter than the standard length for endoscopes built for examination of the colon and intestine and the alimentary canal. Inserting the endoscope guiding device 10 in the endoscope 14 until the handle 50 strikes the upper end 31 of the instrument port 34, as shown in FIG. 5, insures that the entire length of the endoscope 14, except the bending distal tip portion 71, is stiffened for guiding purposes, which results in an easily manipulated and controlled endoscope during insertion into the particular body cavity, regardless of the portion of the endoscope length needed for a particular procedure. In many surgeries, for example in the abdominal wall, much shorter endoscopes are used and properly positioning them is not as arduous as in an examination of the colon, but the endoscope guiding device 10 can easily be designed and manufactured in different lengths, diameters and degrees of stiffness to provide the desired stiffening effect to an endoscope of any given length and inside diameter.

Still referring to FIG. 5, when the endoscope guiding device 10 is inserted into the endoscope 14 until its handle 50 contacts the top 31 of the instrument port 34, often a biopsy port, the endoscope guiding device 10 is automatically inserted into the endoscope 14 to the desired length. The desired length of insertion is short enough to insure that the endoscope stiffening device 10 does not reach the distal end 20 of the endoscope 14, which could lead to punctured colons or other unfortunate problems, but is short enough that a floppy flexible bending tip portion 71, about 8-16 cm, with the preferred length being about 12 cm, of the endoscope 14 remains free from stiffening, and is less stiff because the endoscope guiding device 10 does not enter into the tip portion 71, which is often more flexible than the remaining portion of the endoscope 14 insertion tube itself. Not allowing the endoscope guiding device 10 to enter the flexible bending tip portion 71 has been found to be essential in manipulating the endoscope 14 inside a patient. This specific length requirement prevents using a single endoscope guiding device 10 for endoscopes 14 of different lengths, for having an endoscope stiffening device 10 which is properly located inside the endoscope 14 when the handle 50 contacts the top 31 of the instrument port 34 is a matter of safety. Typically an endoscope 14 for intestinal use is includes an insertion tube portion that is about 166 cm, with the distal end bending tip portion being about 12 cm of that length. Further, at the proximal end of the endoscope 14, the distance from the proximal end of the insertion tube portion to the outer surface of the instrument or biopsy port 34 that the endoscope guiding device 10 is inserted into is about 17 cm long, for a total length of about 183 cm. The corresponding endoscope guiding device 10 is about 170 cm long, resulting in a flexible bending tip portion 71 about 12 cm long. That is, the endoscope guiding device 10 occupies about 91%-96% of the total length of the endoscope 14, with the preferred occupation proportion being about 93.5%, leaving a distal end bending tip portion 71 that is not occupied by the endoscope guiding device 10 that is about 4%-9% of the total length of the endoscope 14, with the preferred proportion being about 6.5%.

In shorter endoscopes, it has been found desirable to provide an endoscope guiding device 10 that does not penetrate a similar distal end portion of the endoscope 14, leaving a distal end portion having a length of about 4%-9% of the total length of the endoscope 14 unoccupied by the endoscope guiding device 10, with length having the meaning defined herein. Typically, the shorter the endoscope 14, the less of the distal end portion needs to remain flexible because a shorter endoscope is inserted a shorter distance into the body and along a straighter path than an endoscope 14 inserted into the colon. In any case there is a distal end portion of the endoscope 14 into which the endoscope guiding device 10 must not be inserted so that the distal end of the endoscope 14 remains flexible and the possibility of perforating the intestine or other body part is eliminated and this distance is typically lies in a range of 4%-9% of the length of the endoscope 12, with the preferred proportion being about 6.5%, that is, the endoscope guiding device 10 penetrates the endoscope 12 a proportionate length of about 91%-96% of the length of the endoscope 12 from the top 31 of the instrument port 34 toward its distal end 20, with the preferred portion being about 93.5%.

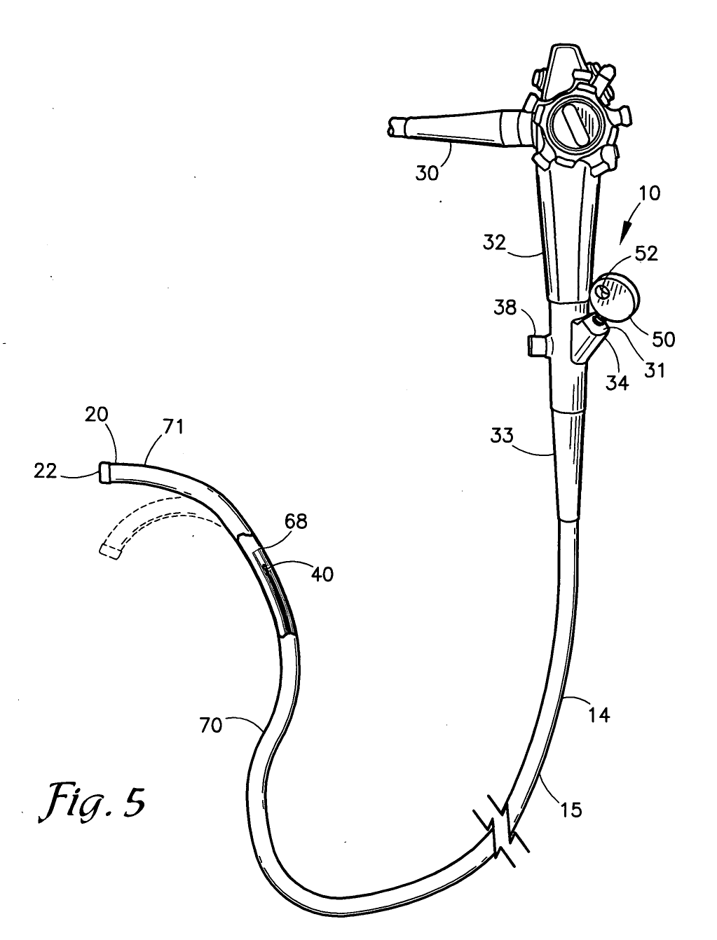

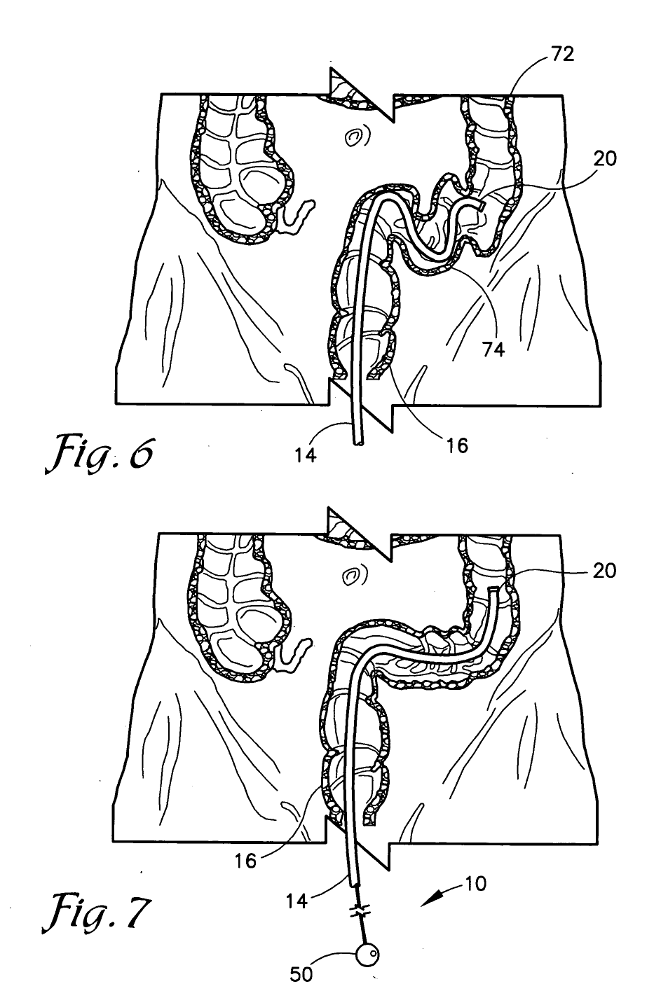

Referring to FIG. 6, an endoscope 14 is being advanced toward the cecum 72 of the intestine 16, but the flexible floppy nature of the endoscope 14 tends to collapse the intestinal track, bunching it up along serpentine path 74.

Referring to FIG. 7, when the endoscope guiding device 10 is inserted into the endoscope body 15 prior to insertion of the endoscope into the intestine 16, the resultant stiffening of the endoscope 14 allows the physician to guide the endoscope body 15 through the intestine 16 and around the naturally occurring folds and bends in the intestine 16 without causing it to bunch up. The physician can position the distal end 20 of the endoscope 14 in the desired location easily and quickly and less skill is required.

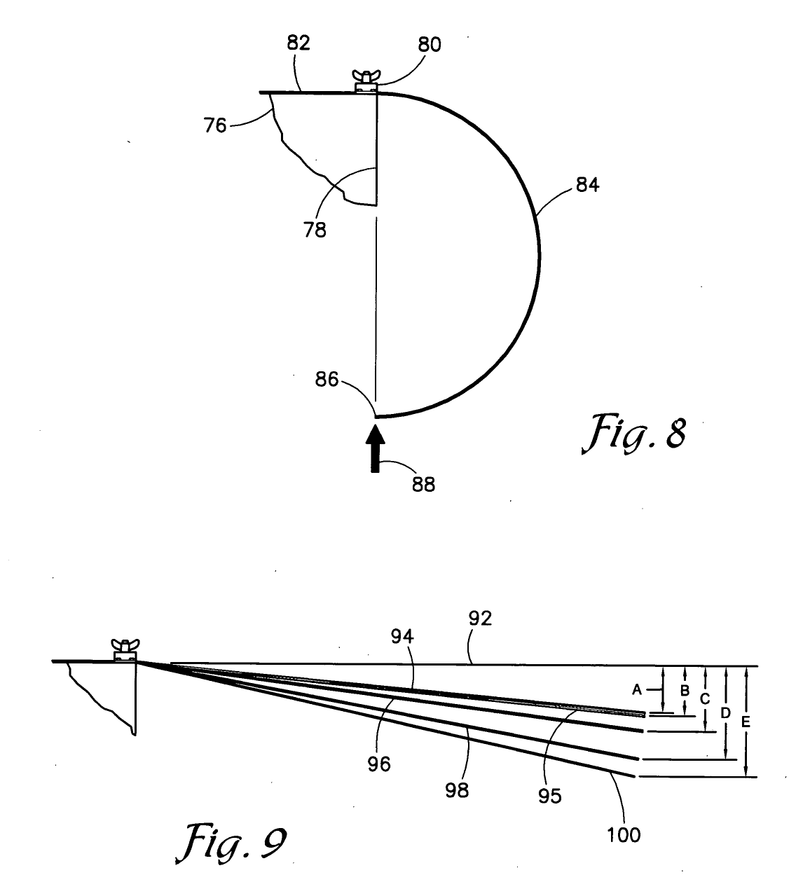

Referring to FIG. 8, the development and selection of the size and material of the wire body 54 of the endoscope guiding device 10 is crucial to the success of the endoscope guiding device 10. It was determined that use of a wire body 54 was desirable and that a wire body 54 composed of a memory wire that would return to its original straight position after any reasonable bending or deformation was desirable because this would allow a physician to begin again with a straight endoscope guiding device 10 for each procedure, regardless of the deformation the wire body 54 may have undergone in a prior procedure. This condition provides the clear advantage of an essentially repeatable user experience for the physician with the principal variable in use of the endoscope guiding device 10 being the variations in intestinal structure from patient to patient.

A suitable base type of memory wire from which to form the endoscope guiding device 10 is a titanium based memory wire, but the specific needs for the present application are not met by the memory wires available commercially and, as these are available in a variety of diameters, it was necessary to device an empirical test to select a wire of the appropriate stiffness. The memory wire of choice for the wire body 54 is nitinol, an alloy of nickel and titanium. In general, nitinol is a family of alloys comprised principally of equiatomic percentages of Nickel and Titanium exhibiting a thermoelastic martensitic transformation to Austenite that creates either shape memory or superelasticity. The name nitinol originated in the 1960s from the chemical symbols Ni (nickel) and Ti (Titanium), plus the initial letters of Navel Ordnance Laboratory, located in Silver Spring, Md., where it was invented. It can be made in a variety of specific formulas and certain other materials are added to enhance specific desired properties, which are typically superelasticity or shape memory in the present application.

For each of these tests, a nitinol wire 61 cm (24 inches) was employed. Still referring to FIG. 8, an appropriate ad hoc test of the relative stiffness of different diameters of basic memory wires utilizes a horizontal work surface 76 having an edge 78 with a free drop off and a screw clamp 80. A proximal end 82 of an appropriate length of basic memory wire test sample 84 is clamped to the horizontal work surface 76 by the screw clamp 80 adjacent to the edge 78, and a measured force required to bend each tested wire downwardly into the 180° of the memory wire 80 as shown wherein the distal end 86 of the test sample 84 lies along a vertical line indicated by the arrow 88 which is aligned immediately adjacent to the edge 78 of the horizontal work surface 82.

The amount of force required to bend the samples into the 180° arc illustrated in FIG. 8 is as follows, using wire of the prescribed types and diameters, with the weight being applied to the distal end tip of each wire:

TABLE 1

| | | | | Deflection |
|---|---|---|---|---|
| Sample # | Wire Type | Diameter-mm | Diameter-in | Force (grams) |
| 1 | 304 SS* | 1.2 mm | (0.048″) | 50.1 g |
| 2 | nitinol | 1.4 mm | (0.055″) | 31.4 g |
| 3 | nitinol | 1.6 mm | (0.063″) | 27.6 g |
| 4 | nitinol | 1.9 mm | (0.073″) | 92.5 g |
| 5 | nitinol | 2.1 mm | (0.083″) | 153.6 g |

*stainless steel

The stainless steel wire was included, in both tests, as a general reference point, related to devices that were experimented with during development of the catheter guiding apparatus 10. As expected, in general, the thicker the wire, the more force was required to bend the wire into a 180° arc. An exception occurs between samples 2, 3, indicating perhaps a different quality of wire through manufacturing variations in the alloy, in heat treating or the like and further indicating the desirability of tighter quality control and of an apparent need for a customized alloy.

Referring to FIG. 9, a second test, determined the amount of deflection below the horizontal reference line 92 caused by the weight of the wire samples 94, 96, 98, 100 themselves was observed. The degree of deflection provides an indication of the stiffness of any particular length of wire, with the particular basic memory wire.

TABLE 2

| | | | | | Downward | Downward | |
|---|---|---|---|---|---|---|---|
| | Ref. | Wire | Diameter - | Diameter - | Deflection - | Deflection - | Ref. |
| Sample # | Char. | Type | mm | in | cm | in | Letter |
| 1 | 94 | 304 SS* | 1.2 mm | (0.048″) | 5.7 cm | (2.250″) | A |
| 2 | 95 | nitinol | 1.4 mm | (0.055″) | 21.6 cm | (8.500″) | B |
| 4 | 96 | nitinol | 1.6 mm | (0.063″) | 25.4 cm | (10.000″) | C |
| 5 | 98 | nitinol | 1.9 mm | (0.073″) | 14 cm | (5.500″) | D |
| 6 | 100 | nitinol | 2.1 mm | (0.083″) | 6 cm | (2.3750″) | E |
| 7* | N/A | nitinol | 1.6 mm | (0.063″) | 21.38 cm | (54.310″) | |

The stainless steel wire was selected as a beginning standard because it imparts a suitable degree of stiffness to an endoscope 14, but, again, is unsuitable because it retains bends and curves. Because nitinol wire does not retain bends, it was believed that this would provide a more suitable stiffening guide, but it tends not to be as stiff as stainless steel wire of the same diameter.

*This sample is not shown in FIG. 9 and is reported here to illustrate the importance of adhering to strict manufacturing specifications for the wire body 54, as this sample was supposed to be the same alloy as used in sample 4, but is clearly far less rigid. In investigating why one sample of 1.6 mm nitinol would provide such different rigidity results, it was determined that different alloys falling into the general category of nitinol have substantially different characteristics important to the endoscope guiding device 10, perhaps most importantly, the rigidity, that is, resistence to bending, and a certain degree of flexibility, as well as the traditional nitinol characteristic of regaining its original shape after moderate deformation.

This result illustrates, as is well known, the critical importance of temperature for creating nitinol with memory shape and superelasticity. A number of thermal points, principally including the fully annealed Austenitic peak and the active Austenitic finish temperatures. The fully annealed Austenitic peak (Aₚ) is the temperature at which the fully annealed nitinol has the highest rate of transformation from Martensite (a body centered cubic form in which some carbon is dissolved) to Austenite (an allotrope having a face centered cubic structure). The Austenitic finish temperature is the temperature at which the material has completely transformed to Austenite, which means that at and above this temperature the material will have completed its shape memory and has completely transformed to Austenite, and the material will therefore have shape memory and superelastic characteristics.

When the material has been fully transformed to Austenite, that material exhibits essentially a density of 6.45 g/cm³; a modulus of elasticity of 75 Gpa; electrical resistivity of 82×10⁻⁶ ohm-cm; a magnetic susceptibility of 3.7×10⁻⁶ emu/g and a coefficient of thermal expansion of 11×10⁻⁶/° C.

Through extensive empirical testing, it has been determined that suitable results are obtained from a 1.6 mm diameter wire body 54 made from nitinol a composition lying in a range of 50-60% Nickel and 50-40% Titanium, with certain trace elements, with the preferred formulation comprising 55%±1% Nickel, with the balance, i.e., 45%±1% Titanium and trace elements of carbon, oxygen, hydrogen and other comprising ≦1%. The trace elements are predominantly carbon and oxygen, with impurity trace elements comprising a small fraction of 1%.

The wire body 54 is further manufactured as a round wire, i.e., having a uniform curricular cross section; and is fully annealed (annealing starting temperature, i.e., Aₛ) at −35° C. to 0° C. with the fully annealed temperature (annealing final temperature, i.e., A_{f}:) being less than 21° C. with a finish as drawn being oxide, bright, or ground finish. The diameter is manufactured to tight tolerances, preferably with tolerance from the specification of not greater than ±0.076 mm (±0.0003 inches). The upper plateau strength is 39.3×10⁸ dynes/cm² (57,000 lbs/in²) maximum with an ultimate tensile strength of 11×10⁹ (160,000 lbs/in²) minimum. The minimum elongation to failure is 6% of the initial length. Suitable results relative to rigidity and to the stiffening effect when used in the endoscope guiding device 10 are achieved by a nitinol wire body 54 adhering to these specifications. This product was specially created and manufactured to order for the endoscope guiding device 10.

The goal of the endeavor that has led to the present endoscope guiding device 10 was to develop a nitinol wire guide that is as stiff as possible, while not requiring excessive force to insert into the endoscope 14. Nitinol was selected due its potential shape retention memory properties. A serious constraint is the diameter of the main channel 45 (FIGS. 2, 3) and the diameter and especially the angle of the sharp turn elbow 44. Endoscopes for exploring the colon or the alimentary canal are available from a number of providers and each provides different endoscopes with different sized channels. The diameter of a wire guide cannot equal or exceed the diameter of the main channel or the elbow fitting where a stiffening guide would typically be inserted. Therefore, the maximum diameter of a wire guide cannot exceed about 2.1 mm, but it is desired to used the endoscope guiding device 10 with any conventional channel diameters. If, however, the wire guide is larger than about 1.7-1.8 mm, it is so stiff and so close to the diameter of the elbow 44 and main channel 45 that insertion into the endoscope 14 requires so much force that a normal physician must use both hands to push on the wire guide and therefore requires an assistant to hold the endoscope 14 during insertion of the endoscope guiding device 10. Further, insertion of such a thick wire guide very likely damages the endoscope 14 or shortens its life by abrading the interior side wall of the endoscope 14.

Through empirical tests conducted by a physician experienced in this field, it was determined that 1.6 mm diameter nitinol wire provided the best characteristics of providing a smooth and natural feel during insertion and withdrawal from the endoscope 14 and a suitable degree of stiffening of the endoscope 14, but that initial insertion into the port 36 was excessively difficult and provided too much resistance. It was further determined that a nitinol wire of suitable alloy composition of about 1.2 mm provides a suitable feel and force for insertion into and withdrawal from the endoscope 14, but is not stiff enough to provide a sufficient stiffening effect to the endoscope. Therefore, it was desired to combine the desirable insertion and withdrawal characteristics of a relatively thin wire with the stiffening effects of a thicker wire.

It was determined that this could be accomplished by making a portion of the wire body 54 adjacent to the of the distal end of the wire body 54 weaker and therefore more flexible than the remaining portion of the wire body 54. This function can be accomplished through a number of different methods. For example, a length adjacent to the distal end 40 can be selectively heat treated or annealed to be more flexible than the remaining portion of the wire body 54 formed from a thinner wire through an extrusion process, and so forth. Because nitinol wire cannot be welded, it is not feasible to provide a memory wire fashioned from two different diameter wires welded or otherwise securely fastened together.

In the preferred embodiment, the distal end portion of the wire body 54 is formed into a reduced diameter portion by grinding, preferably centerless grinding, with a tapered transition zone between the reduced diameter portion and the remainder of the wire body 54 providing a gradual transition from a larger diameter to a smaller diameter to prevent formation of a sharp stepwise transition that would produce a frangible line and catch or hinder insertion. If the distal end portion of the wire body 54 were to break at a stepwise transition while the endoscope guiding device 10 were inside an endoscope 14, the endoscope 14 could be damaged or ruined and the procedure on a patient would be delayed.

Referring to Figs, 10, 11, the distal end 40 includes a rounded tip 42 formed by centerless grinding the distal end 40 of the wire body 54, and adjacent to the distal end 40 and moving upwardly toward the proximal end lies a reduced diameter portion 102 having parallel edges, which is followed by a transition zone 104 to the reduced diameter portion 102, which consists of a uniform taper along the transition zone 104 from the wire body 54 full diameter at 106 to the reduced diameter portion 102. The transition zone 104, the reduced diameter portion 102 are formed by grinding and naturally results in the rounded tip 42, as the movement of the grinder is halted prior to reaching the exact end of the wire body 54, principally to prevent grinding off a portion of the tip of the wire body 54.

The rounded tip 42 at the distal end 40 is essentially hemispherical and has the same radius as the unground portion of the wire body 54, with an accuracy of about 0.0025 mm (0.001 inch)

Grinding the diameter of the wire body 54 adjacent to the distal end 40, or otherwise reducing the diameter of the wire body 54, weakens the wire body 54 , thereby reducing its stiffness sufficiently to facilitate inserting the distal end 40 into the endoscope 14 port 34 enough to provide the physician user with a comfortable consistent feel compared with the feel of inserting and withdrawing medical the instruments into and out of endoscopes and to ease insertion of the endoscope guiding device 10 into the endoscope 14, while the rounded tip 42 prevents damage to the interior side wall 46 of the endoscope 44 at any point along the endoscope 14. The reduced diameter distal end portion of the endoscope guiding device also improves mobility and flexibility of the endoscope 14, allowing the physician to manipulate the endoscope 14 inside the patient more surely.

The same technique described immediately above is also advantageously used to weaken a wire body 54 at any desired location along its length to increase its flexibility at any location between its proximal end 48 and its distal end 40.

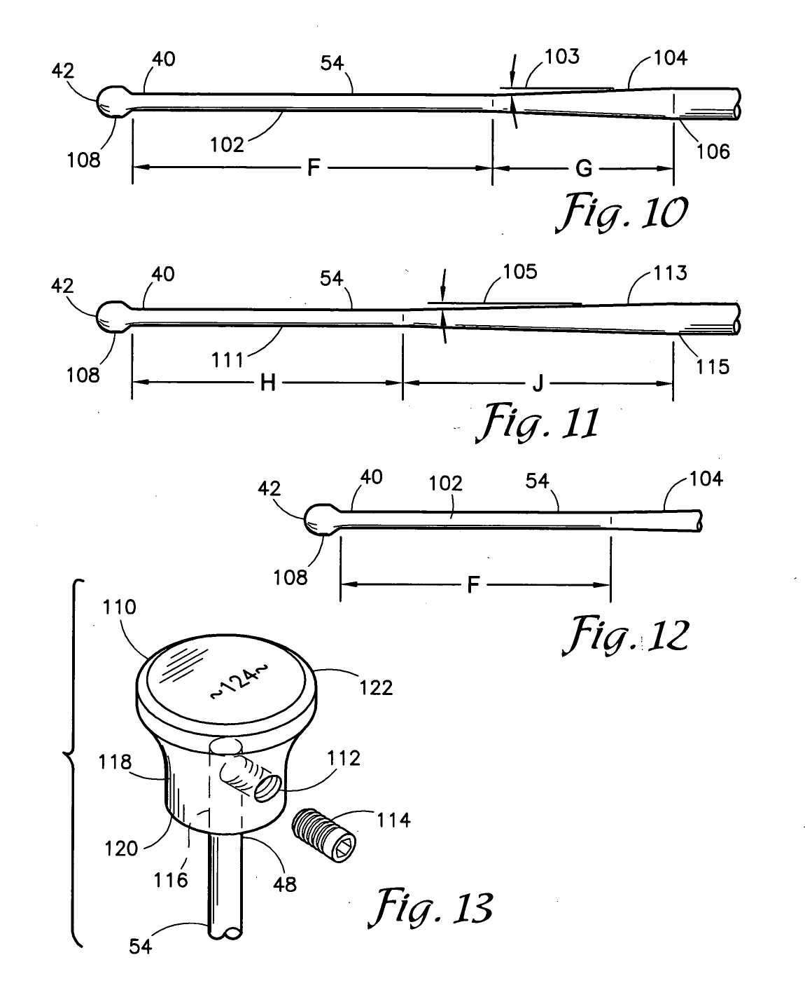

Still referring to FIG. 10, the reduced diameter portion 102 has a uniform diameter of 1.2 mm and a length F lying within a range of about 1.2 cm (0.5 inches) to 3.3 cm (1.25 inches), with the preferred length F being about 2 cm (0.75 inches), with the objective being to provide a thinner more flexible portion long enough so that it is about the same length of the path of the sharp elbow turn 44 of the endoscope 14 so that the distal end 40 of the wire body 54 rounds the elbow turn 44 and moves into a position tangent to the elbow turn 44 and then turns straight down the main channel 45 before the stiffer thicker diameter portion of the wire body 54 enters into the upper end 31 instrument port 34. That is, the reduced diameter portion 102 guides the thicker portion of the wire body 54 into the endoscope 14. The transition zone 104 is ground down along a uniform taper at an angle 103 lying in a range of about 0.5° to 3°, with the preferred angle being about 2° over a distance lying in a range of about 1.2 cm (0.5 inches) to 3.3 cm (1.25 inches), with the preferred length G being about 2 cm (0.75 inches) and the remaining portion of the wire body having a diameter lying in a range of about 1.4-1.9 mm, with the preferred thickness being about 1.6 mm. The angle of the tapered section will be ultimately determined by the length of the transition zone, the beginning diameter, that is, the diameter of the main portion of the wire body 54, and the diameter of the reduced diameter portion 102.

Referring to FIG. 11, in an alternative embodiment of a suitable endoscope guiding device 10 has a wire body 54 having a reduced diameter section 111 has a uniform diameter of 1.2 mm and a length H of lying within a range of about 1.2 cm (0.5 inches) to 3.3 cm (1.25 inches), with the preferred length H being about 2 cm (0.75 inches); a transition zone 113, having a length J having uniform taper at an angle 105 lying in a range of about 0.5° to 5°, with the preferred angle being about 2° over a distance lying in a range of about 1.2 cm (0.5 inches) to 3.3 cm (1.25 inches), with the preferred length J being about 2 cm (0.75 inches) and the remaining portion of the wire body having a diameter lying in a range of about 1.4-2.2 mm, with the preferred thickness being about 1.9 mm, an embodiment providing greater stiffness than the embodiment shown in FIG. 10, assuming the same alloy. The transition zones 103, 105 are designed to provide a gradual transition from the thicker portion of the wire body 54 to the thinner end of the reduced diameter easy insertion portion 102, 111 to avoid a sharp drop off in diameter, which would provide a point of exaggerated weakness that would be more subject to breakage and catching or jamming on insertion.

Referring to FIG. 12, the enlarged view of the distal end of the wire body 54 of FIG. 10 more clearly illustrates the relatively flat tip end portion 108 of the rounded end 42.

Referring to FIG. 13, an alternative embodiment of a handle for the endoscope guiding device 10 includes a knob 110 having a horizontal threaded bore 112 for accepting the set screw 114 and a vertical bore 116 for receiving the proximal end 48 of the wire body 54, which is clamped into position in the vertical bore 116 by the set screw 114. The knob 110 includes a basically cylindrical body 118 that swells upwardly from the bottom edge 120, providing an enlarged gripping ledge portion 122 adjacent to the top 124. A handle such as the knob 110 or the disk-shaped handle 50 that clamps the wire body 54 using a set screw has proved useful and desirable because nitinol cannot be welded readily. Alternatively, however, the wire body 54 may be secured to the handle 50 or knob 110 with a suitable adhesive.

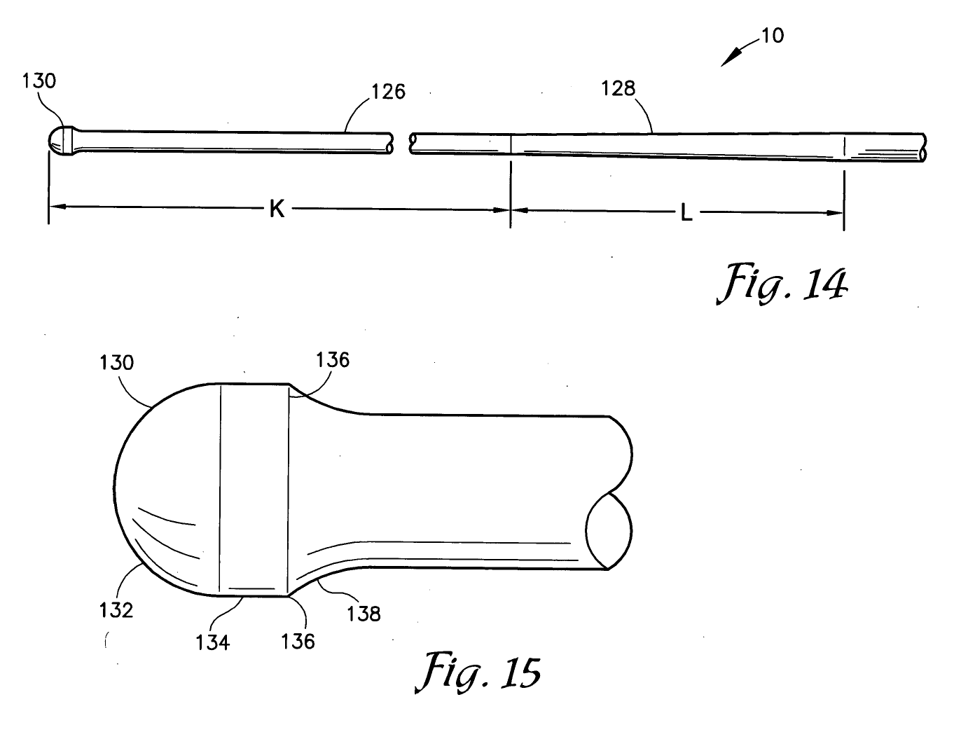

Referring to FIG. 14, another embodiment of the endoscope guiding device 10 includes a distal end reduced diameter portion 126 having a length K that is longer than the reduced diameter distal end portions shown in FIG. 10, 11, which is about 10 cm (4 inches) long, which is immediately adjacent to the transition zone 128 having a length L, which is about 0.5 cm (0.635 inches) long. It has been found that a longer reduced diameter portion K provides increased mobility and flexibility in the distal end portion 40 of the endoscope guiding device 10, that facilitates such manipulation of the endoscope 14 by the physician, leading to quicker and easier movement of the endoscope 14 through the body and to quicker, easier and more accurate insertion into a desired target. The diameter of the reduced diameter portion L and the diameters and taper of the transition zone L are as described above in connection with FIGS. 10-12.

Referring to FIG. 15, the rounded distal end tip 130 of the embodiment of FIG. 14, includes a substantially hemispherical tip end portion 132 having a radius equal to the unworked radius of the wire body 54, that is about 0.8 mm adjacent to a unworked band portion 134 having a diameter of about 1.6 mm having a right-hand edge 136 that falls off at the concave radius cut 138 of about 0.000254 mm.

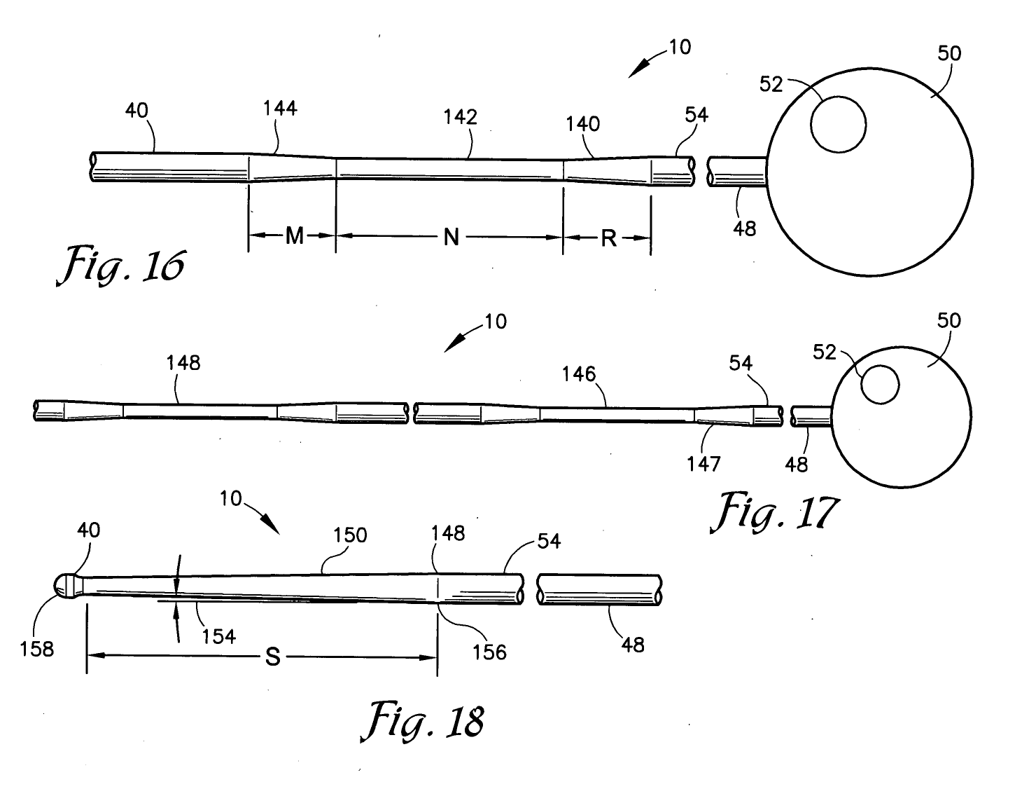

Referring to FIG. 16, in some applications, for example, in negotiating the kink 19 (FIG. 1), it is useful to have a section of decreased strength, that is, increased flexibility at some location along the wire body 54 between the distal end 40 and the proximal end 48, which is achieved by providing a reduced diameter portion 142 over any desirable length N, for example 10-20 cm (4-8 inches) with a proximal transition zone 140 of a length R, about 0.5 cm (0.635 inches) and a distal transition zone 144 of a length M, about 0.5 cm (0.635 inches). Each transition zone 140, 140 has a taper as described in the discussion of FIGS. 10-12, each of which tapers inwardly toward the reduced diameter portion 142. This configuration can be formed onto the device of FIG. 14, that is, with a distal end reduced diameter portion, or may be formed into an otherwise uniform diameter wire body 54.

Referring to FIG. 17, a wire body 54 may be desirably equipped with two intermediate reduced diameter sections 146, 148, each having tapered transition zones, diameters, and section lengths as described above. The thinner diameters of the reduced diameter sections shown in all the FIGS. provide a weakened portion of the wire body 54 that makes it more flexible along the full length of the reduced diameter portion. These weakened reduced diameter portions 146, 148 can be formed by centerless grinding or the like and can be any desired length of the whole length of the wire body 54 of the endoscope guiding device. Similarly, the transition zone may be any desired length.

Referring to FIG. 18, because in most applications the difference in diameter of the wire body 54 and any reduced diameter portion is quite small, e.g., 1.6 mm−1.1=0.5 mm and the taper in a transition zone is about 1.5°-2.5°, with the preferred angle being about 2°, and producing the reduced diameter portion and rounded distal end tip 42, 130 adds another step to the manufacturing process, it is sufficient to provide a uniform taper zone 150 having a length S lying an a range from about 1cm-20 cm (0.5-8 inches), with a uniform taper 154 lying in a range of 0.5°-3°, with the preferred angle being about 1.5°, with the taper beginning at a point 156 toward the proximal end 48 of the wire body 54 and proceeding along a continuous uniformly tapered diameter to the distal end rounded tip 158, which is the rounded tip described above.

While the present invention has been described in accordance with the preferred embodiments thereof, the description is for illustration only and should not be construed as limiting the scope of the invention. Various changes and modifications may be made by those skilled in the art without departing from the spirit and scope of the invention as defined by the following claims.

We claim:

1. A device comprising a wire body having a proximal end and a distal end and means for reducing the stiffness of said wire body of at at least one predetermined location along said wire body.

2. A device in accordance with claim 1 wherein said stiffness reducing means further comprises a reduced diameter portion of said wire body at said at least one predetermined location along said wire body.

3. A device in accordance with claim 2 further comprising a transition zone between said reduced diameter portion and at least one remaining portion of said wire body.

4. A device in accordance with claim 3 wherein said transition zone further comprises a uniform taper having an angle lying in the range of 0.5°-5° inwardly of the full diameter of said wire body to said reduced diameter portion.

5. A device in accordance with claim 1 further comprising a wire body made of nitinol.

6. A device in accordance with claim 5 wherein said nitinol of said wire body comprises an alloy including Nickel in a range of 50%-60% and about 50%-60% Titanium and less than or equal to 1% trace elements.

7. A device in accordance with claim 6 wherein said nitinol consists of 55%±1% Nickel, 45%±1% Titanium and trace elements of carbon, oxygen and other comprising ≦1%.

8. A device in accordance with claim 1 further comprising a handle fastened to said proximal end of said wire body.

9. A device in accordance with claim 8 wherein said handle further comprises a handle body having a bore for receiving said proximal end of said wire body and means for fixing said proximal end of said wire body in said bore.

10. A device in accordance with claim 1 wherein said reduced diameter portion has a length sufficient for said distal end to enter a main channel of an endoscope prior to introduction of a full diameter portion of said wire body.

11. A device in accordance with claim 1 further comprising a handle fixed to a proximal end of said wire body and said wire body has a length such that when a proximal end of said wire body is inserted into an endoscope until said handle contacts an outer surface of an instrument port of said endoscope said distal end of said wire body penetrates a channel of said endoscope to a particular desired point.

12. A device in accordance with claim 11 wherein said distal end of said wire body penetrates said endoscope from a proximal end of said endoscope toward a distal end of said endoscope a distance lying in the range of 91%-96% of the length of said endoscope.

13. A device comprising wire body having a proximal end and a distal end and a reduced diameter portion adjacent to said distal end a tapered transition zone between said reduced diameter portion and the remaining length of said wire body.

14. A device in accordance with claim 13 further comprising a rounded tip on said distal end.

15. A device in accordance with claim 1 wherein said stiffness reducing means further comprises a uniform inward taper adjacent to said distal and of said wire body.

16. A device in accordance with claim 13 wherein said transition zone further comprises a uniform taper having an angle lying in the range of 0.5°-5° inwardly of the full diameter of said wire body to said reduced diameter portion.

17. A device in accordance with claim one wherein said stiffening reducing means further comprises a first reduced diameter portion of said wire body adjacent to said distal end of said wire body and a second reduced diameter portion of said wire body at a location between said proximal and of said wire body and said first reduced diameter portion of said wire body.

18. A device in accordance with claim 1 further comprising a rounded distal end tip on said wire body, said round distal end tip having a flat portion having a diameter of the natural diameter of said wire body adjacent to a reduced diameter portion of said wire body adjacent to said round distal end tip.

19. A device comprising wire body having a proximal end and a distal end and a reduced diameter portion adjacent to said distal end and a tapered transition zone between said reduced diameter portion and the remaining length of said wire body.

20. A device in accordance with claim 19 further comparing a reduced diameter portion having a length lying in a range of 1.2 cm (0.5 inches) to 3.3 cm (1.25 inches) and said tapered transition zone has a length lying in a range of 1.2 cm (0.5 inches) to 3.3 cm (1.25 inches) and a uniform taper lying in a range of 0.5°-5° inwardly of the full diameter of said wire body to said reduced diameter portion.
